(19) United States
(12) Patent Application Publication
(10) Pub. No.: US 2015/0232398 A1
(43) Pub. Date: Aug. 20, 2015

(54) AZULENE AND DERIVATIVES THEREOF AS COLORANTS

(71) Applicant: LUDWIG-MAXIMILIANS-UNIVERSITÄT MÜNCHEN, München (DE)

(72) Inventors: Heinz Langhals, Ottobrunn (DE); Moritz Eberspächer, Munich (DE)

(21) Appl. No.: 14/433,708

(22) PCT Filed: Oct. 8, 2013
(86) PCT No.: PCT/EP2013/070930

§ 371 Date: Apr. 6, 2015

(30) Foreign Application Priority Data
Oct. 9, 2012 (DE) 10 2012 019 843.4
Mar. 7, 2013 (DE) 10 2013 003 915.0

(51) Int. Cl. C07C 13/52; A61K 47/10; A61K 31/025; A61K 47/24; A61K 47/06

(52) U.S. Cl. CPC C07C 13/52; A61K 47/24; A61K 47/06; A61K 31/025; A61K 47/10; C07C 2102/30

(57) ABSTRACT

Disclosed is the use of azulene or azulene derivatives for coloring compounds selected from siloxanes and perfluorinated compounds, and compositions that contain azulene or an azulene derivative together with a siloxane or a perfluorinated compound.

Polysiloxanes, such as polyorganosiloxanes or silicones, are characterized by a high degree of thermal stability and are largely resistant to chemicals, radiation and oxidizing agents (cf. e.g. P. Kunststoffe—Eigenschaften and Anwendungen [plastic materials—properties and applications], 7^{th} revised and extended edition, Springer publishing house, Berlin, 2008). Silicones are commercially available as silicone oil, silicone rubbers and silicone resins, and are used in a variety of applications, inter alia in food technology, cosmetics and medicine.

Perfluorocarbon compounds are hydrocarbon compounds wherein all the hydrogen atoms have been replaced with fluorine atoms. Compared to their hydrogen analogues, they are characterized by a particularly high degree of physical, chemical and thermal stability. Furthermore, perfluorocarbon compounds are nonflammable and physiologically inert (cf. e.g. Ullmann's Encyclopedia of Industrial Chemistry, 6^{th} edition, volume 14, Wiley-VCH publishing house, Weinheim, 2003). Similar properties are also known for other perfluorinated compounds such as perfluoroamines or perfluoroethers.

Polysiloxanes and fluoroorganic compounds are often used in applications requiring chemically inert behavior. Therefore, it is also difficult to provide colorants which are able to interact with such compounds, e.g. by forming a stable solution, and thus coloring them. It is the object of the present invention to provide a colorant which can solve this problem.

Within in the framework of the present invention, it was found that due to their surprisingly high solubility both in polysiloxanes and in fluoroorganic compounds, compounds with an azulene basic structure, which absorb light in the visible spectral range, can be used as colorants for those compounds. Azulene of formula (I) as well as azulene derivatives are valued for their anti-inflammatory effects (cf. (a) A. E. Sherndal, J. Am. Chem. Soc. 1915, 37, 1537-1544; (b) L. Ruzicka, E. A. Rudolph, Helv. Chim. Acta 1926, 9, 118-140. (c) A. Pfau, P. Plattner, Helv. Chim. Acta 1936, 19, 858-879; (d) P. Plattner, Helv. Chim. Acta 1941, 24, 283-294). Such compounds are obtained in particular as active ingredients from chamomile.

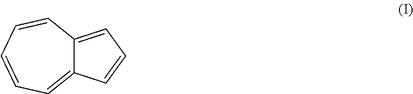

Azulene (I) has a deep blue hue. Furthermore, azulene is especially characterized by low toxicity (M. Struwe, M. Csato, T. Singer, E. Gocke, Mutation Res., Genetic Toxicol, and Environmental Mutagenesis 2011, 723, 129-133. (b) R. Teufel, Int. J. Toxicol. 1999, 18 (Suppl. 3), 27-32. (c) L. I. Sweet, P. G. Meier, Bull. Environment. Contain in. and Toxicol. 1997, 58, 268-274).

Since it is an aromatic hydrocarbon, one would expect azulene to be highly soluble in lipophilic media such as toluene or isohexane, which is the case. The present inventors furthermore found a surprisingly universal solubility of azulene (I) which shows significant solubility even in the above-mentioned inert classes of compounds.

Thus, a first aspect of the present invention is directed to the use of a compound of the general formula (II):

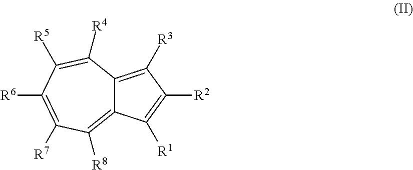

for coloring a compound selected from a siloxane, a perfluorocarbon compound, a perfluoroether and a perfluoro amine,
wherein in formula (II) the groups R¹ to R⁸ can be the same or different and are independently selected from a hydrogen atom, a halogen atom, a cyano group, a linear alkyl group, a group —(CH₂)ₙ-phenyl, a group —(CH₂)ₙ-pyridyl, a group —(CH₂)ₙ-thiophene, wherein n is an integer from 0 to 6, a naphthalene group, wherein one or two CH groups can be replaced with nitrogen atoms, or an anthracene group, wherein one or two CH groups can be replaced with nitrogen atoms,
wherein the linear alkyl group is an alkyl group with at least one and at most 37 C-atoms, wherein one to 10 CH₂ units can independently be replaced each with a carbonyl group, an oxygen atom, a sulfur atom, a selenium atom, a tellurium atom, a cis- or trans-CH═CH group, wherein one CH unit can also be replaced with a nitrogen atom, an acetylenic group, a divalent phenyl group (e.g. a 1,2-, 1,3- or 1,4-phenyl group), a divalent pyridine group (e.g. a 2,3-, 2,4-, 2,5-, 2,6-, 3,4- or 3,5-pyridine group), a divalent thiophene group (e.g. a 2,3-, 2,4-, 2,5- or 3,4-thiophene group), a divalent naphthalene group (e.g. a 1,2-, 1,3-, 1,4-, 1,5-, 1,6-, 1,7-, 1,8-, 2,3-, 2,6- or 2,7-naphthalene group), wherein one or two CH groups can be replaced with nitrogen atoms, or a divalent anthracene group (e.g. a 1,2-, 1,3-, 1,4-, 1,5-, 1,6-, 1,7-, 1,8-, 1,9-, 1,10-, 2,3-, 2,6-, 2,7-, 2,9-, 2,10- or 9,10-anthracene group), wherein one or two CH groups can be replaced with nitrogen atoms,
and wherein up to 12 individual hydrogen atoms of the CH₂ groups in an alkyl group can independently be replaced also on the same C-atom with a halogen atom, a cyano group, or a linear alkyl chain with up to 18 carbon atoms, wherein one to 6 CH₂ units can independently be replaced each with a carbonyl group, an oxygen atom, a sulfur atom, a selenium atom, a tellurium atom, a cis- or trans CH═CH group, wherein one CH unit can also be replaced with a nitrogen atom, an acetylenic C≡C group, a divalent phenyl group (e.g. a 1,2-, 1,3- or 1,4-phenyl group), a divalent pyridine group (e.g. a 2,3-, 2,4-, 2,5-, 2,6-, 3,4- or 3,5-pyridine group), a divalent thiophene group (e.g. a 2,3-, 2,4-, 2,5- or 3,4-thiophene group), a divalent naphthalene group (e.g. a 1,2-, 1,3-, 1,4-, 1,5-, 1,6-, 1,7-, 1,8-, 2,3-, 2,6- or 2,7-naphthalene group), wherein one or two CH groups can be replaced with nitrogen atoms, or a divalent anthracene group (e.g. a 1,2-, 1,3-, 1,4-, 1,5-, 1,6-, 1,7-, 1,8-, 1,9-, 1,10-, 2,3-, 2,6-, 2,7-, 2,9-, 2,10- or 9,10-anthracene group), wherein one or two CH groups can be replaced with nitrogen atoms, and wherein up to 12 individual hydrogen atoms of the CH₂ groups in the alkyl chain which can serve as the substituent at the alkyl group can independently be replaced also on the same C-atom each with a halogen atom, a cyano group, or a linear alkyl chain with up to 18 C-atoms, wherein one to 6 CH₂ units can independently be replaced each with a carbonyl group, an oxygen atom, a sulfur atom, a selenium atom, a tellurium atom, a cis- or trans-CH═CH group, wherein one CH unit can also be replaced with a nitrogen atom, an acetylenic C≡C group, a divalent phenyl group (e.g. a 1,2-, 1,3- or 1,4-phenyl group), a divalent pyridine group (e.g. a 2,3-, 2,4-, 2,5-, 2,6-, 3,4- or 3,5-pyridine group), a divalent thiophene group (e.g. a 2,3-, 2,4-, 2,5- or 3,4-thiophene group), a divalent naphthalene group (e.g. a 1,2-, 1,3-, 1,4-, 1,5-, 1,6-, 1,7-, 1,8-, 2,3-, 2,6- or 2,7-naphthalene group), wherein one or two CH groups can be replaced with nitrogen atoms, or a divalent anthracene group (e.g. a 1,2-, 1,3-, 1,4-, 1,5-, 1,6-, 1,7-, 1,8-, 1,9-, 1,10-, 2,3-, 2,6-, 2,7-, 2,9-, 2,10- or 9,10-anthracene group), wherein one or two CH groups can be replaced with nitrogen atoms,
and wherein two linear alkyl groups as defined above and located at different positions R¹ to R⁸ can optionally be linked to each other forming a ring.

The variable n is preferably an integer from 0 to 2, in particular 0 or 1.

Unless specifically defined otherwise, the term halogen represents F, Cl, Br and I, in particular F, CI and Br.

The linear alkyl group as an option for R¹ to R⁸, which can be substituted as defined above and wherein one or more CH₂ groups can be substituted, is preferably an alkyl group with at least one and at most 37 C-atoms, wherein one to 10 CH₂ units can independently be replaced each with an oxygen atom, a sulfur atom, a cis- or trans-CH═CH group, wherein one CH unit can also be replaced with a nitrogen atom, a divalent phenyl group, a divalent pyridine group or a divalent thiophene group,
wherein up to 12 individual hydrogen atoms of the CH₂ groups in an alkyl group can independently be replaced also on the same carbon atom with a linear alkyl chain with up to 18 C-atoms, wherein one to 6 CH₂ units can independently be replaced each with an oxygen atom, a sulfur atom, a cis- or trans-CH═CH group, wherein one CH unit can also be replaced with a nitrogen atom, a divalent phenyl group, a divalent pyridine group, or a divalent thiophene group, and wherein up to 12 individual hydrogen atoms of the CH₂ groups in the alkyl chain which can serve as the substituent at the alkyl group can independently be replaced also on the same C-atom each with a linear alkyl chain with up to 18 carbon atoms, wherein one to 6 CH₂ units can independently be replaced with an oxygen atom, a sulfur atom, a cis- or trans-CH═CH group, wherein one CH unit can also be replaced with a nitrogen atom, a divalent phenyl group, a divalent pyridine group, or a divalent thiophene group.

An especially preferred linear alkyl group as an option for R¹ to R⁸ as defined above is an alkyl group with at least one and at most 6 C-atoms, wherein one to three CH₂ units can independently be replaced each by an oxygen atom, a sulfur atom, or a cis- or trans-CH═CH group,

wherein up to three individual hydrogen atoms of the CH₂ groups in an alkyl group can independently be replaced also on the same carbon atom each with a linear alkyl chain with up to 6 carbon atoms, wherein one to three CH₂ units can independently be replaced each with an oxygen atom, a sulfur atom, or a cis- or trans-CH═CH group, and wherein up to three individual hydrogen atoms of the CH₂ groups in the alkyl chain which can serve as the substituent at the alkyl group can independently be replaced also on the same carbon atom each with a linear alkyl chain with up to 6 carbon atoms, wherein one to three CH₂ units can independently be replaced each with an oxygen atom, a sulfur atom, or a cis- or trans-CH═CH group.

With respect to the option according to which two linear alkyl groups as defined above and located at different positions R¹ to R⁸ can be linked to each other forming a ring, it is preferred that the ring be formed by two adjacent groups R¹ to R⁸. Together with the carbon atoms to which the respective two groups are bonded, such a ring preferably comprises 5 to 7 ring members, especially preferred 5 or 6 ring members. Carbocyclic rings and rings which comprise one oxygen atom in addition to carbon atoms are preferred.

A ring which is optionally formed by two groups R¹ to R⁸ is annelated with the azulene basic structure of formula (II). It can be aromatic, but it can also comprise one or more C—C double bonds which are not conjugated with the aromatic system of the azulene basic structure, or it can comprise no further double bonds in addition to those in the azulene basic structure.

Preferably, the groups R¹ to R⁸ in formula (II) are the same or different and are independently selected from a hydrogen atom, a halogen atom, a cyano group and a linear alkyl group which can be substituted as described above and wherein one or more CH₂ groups can be replaced, and wherein two linear alkyls groups as defined above and located at different positions R¹ to R⁸ can optionally be linked to each other forming a ring.

More preferred, the groups R¹ to R⁸ in formula (II) are the same or different and are independently selected from a hydrogen atom and a linear alkyl group, wherein the linear alkyl group is as defined above.

Taking into account the general and preferred definitions given above, it is especially preferred that the groups R¹ to R⁸ in formula (II) be independently selected from a hydrogen atom, C₁-C₆ alkyl, or (C₁-C₆ alkyl)-O—, wherein the term “alkyl” in this connection stands for a linear or branched unsubstituted alkyl unit without the option of substituting a CH or CH₂ unit comprised therein. Compounds of formula (II) wherein the groups R¹ to R⁸ are independently selected from a hydrogen atom and C₁-C₆ alkyl, wherein at most three of the groups R¹ to R⁸ can be alkyl, are especially preferred.

The most preferred compound of formula (II) is azulene, i.e. the compound of formula (I):

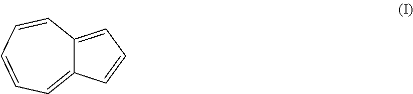

Within the framework of the present invention, a single compound of formula (II) or a compound of formula (I) can be used by itself. However, if needed, two or more different compounds of formula (II) can be used in admixture.

According to the present invention, the compound of formula (II), in particular azulene, is used to color a compound selected from a siloxane, a perfluorocarbon compound, a perfluoroether and a perfluoro amine.

The compounds to be colored with a compound of formula (II) are usually solids or liquids, typically at a temperature of 20° C. and a pressure of 1 atm (101325 Pa).

With the help of the compound(s) of formula (II), individual compounds or mixtures of different compounds can be colored.

Siloxanes, which can be used according to the present invention, are generally compounds which comprise the siloxane bond (Si—O—Si). The term comprises siloxanes wherein the free valences of the silicon atoms are saturated with hydrogen, as well as siloxanes wherein the free valences of the silicon atoms are saturated with organic groups such as alkyl or aryl groups. The latter are also referred to as organosiloxanes. In particular, polysiloxanes, including polyorganosiloxanes (silicones) are also encompassed as compounds. Polysiloxanes and polyorganosiloxanes typically comprise repeating units of the structure (III):

—[SiR^{a}R^{b}—O]— (III),

wherein R^{a} and R^{b} are selected from hydrogen, an organic groups such as an alkyl group (e.g. C₁-C₆ alkyl, in particular methyl) or an aryl group (e.g. phenyl). Typically, the silicones only carry organic groups R^{a} and R^{b}.

Polysiloxanes, and in particular silicones, can have a linear structure, a cyclic structure, a branched structure or a cross-linked structure. Branched and cross-linked structures can be formed, as the person skilled in the art is aware, with units wherein more than one oxygen atom is assigned to each silicon atom. They are also referred to as [T] units (e.g. R^{a}SiO_{3/2}) or [Q] units (e.g. SiO_{4/2}).

Preferred silicones are silicones with repeating units of the following structures (IV) and/or (V):

—[Si(CH₃)₂—O]— (IV)

—[Si(Ph)(CH₃)—O]— (V),

wherein Ph represents a phenyl group.

Surprisingly, the compounds of formula (II) are not only suitable for coloring short-chain siloxanes, such as hexamethyldisiloxane, but are also soluble in sufficient amounts in silicones with higher molecular weights, such as solid silicone or silicone oil, to provide them with an intense color which is easily perceived visually.

Also as silicone oils, both cyclic and linear silicone oils may be used. For instance, the compounds of formula (II) can be used to color common commercially available silicon oils, such as e.g. linear silicone oils with a kinematic viscosity in the range of 0.65 to 1.000.000 mm²/s, and in particular of 10 to 500.000 mm²/s, at 25° C. (measured according to DIN 53019).

The coloring of perfluorinated materials, such as e.g. perfluorinated liquids, works also well. This is particularly surprising since such materials are, inter alia, used specifically when media are needed which should basically not interact with any other substances.

Perfluorocarbon compounds which can be used according to the present invention are compounds which are only formed from carbon and fluorine. They encompass, in particular, perfluoroalkanes, perfluorocycloalkanes, perfluoroolefins and perfluoroaromatics. Compounds with 4 to 20 carbon atoms, or, in the case of cyclic compounds or compounds with a cyclic unit, compounds with 5 to 20 atoms are preferred. In addition, compounds which are liquid at 20° C. and 1 atm (101325 Pa) are preferred.

Perfluoroalkanes are linear or branched alkanes wherein all the hydrogen atoms are replaced with fluorine atoms. Perfluoroalkanes which can well be used in the present invention are in particular perfluoroalkanes with 4 to 20, in particular 5 to 12, carbon atoms. Perfluorohexane, perfluoroheptane, perfluorooctane, perfluorononane or perfluorodecane are preferred.

Perfluorcycloalkanes are alkanes comprising a cyclic unit, wherein in addition one or more linear or branched alkyl chains can be linked to the cyclic unit and wherein all the hydrogen atoms at the cyclic units and the optional linear or branched alkyl chains are replaced with fluorine atoms. Examples of preferred perfluorocycloalkanes for use in the present invention include perfluoromethylcyclohexane, perfluoroethylcyclohexane, or perfluorodecalin.

Perfluoroolefins are compounds which comprise at least one olefinic carbon-carbon double bond and wherein, compared to the olefin as the parent compound, all the hydrogen atoms have been replaced with fluorine atoms. A perfluoro olefin suitable for use in the present invention is for example perfluoro-2-methyl-2-pentene.

Perfluoroaromatics are aromatics wherein all the hydrogen atoms have been replaced with fluorine atoms. Perfluoroaromatics suitable for use in the present invention are for example perfluorobenzene, perfluoronaphthalene or perfluorophenanthrene.

Perfluoroamines which can be used in the present invention are in particular perfluorocarbon compounds which additionally comprise at least one amine function —NR^{c}R^{d}, wherein R^{c} and R^{d} are independently hydrogen or a perfluorocarbon group or wherein R^{c} and R^{d} can be linked to each other forming a ring. Preferably, the groups R^{c} and R^{d} are both perfluorocarbon groups, in particular perfluoroalkyl groups. Perfluoroamines with a perfluoroalkane structure which comprises one or more tertiary amine functions as additional amine function(s) are especially preferred. The number of carbon atoms in the perfluoroamines is also preferably in the range of 4 to 20, more preferred 5 to 12.

Perfluoroethers which can be used in the present invention are in particular perfluorocarbon compounds which additionally comprise at least one ether bond (C—O—C). In particular, the term also encompasses perfluoropolyether, i.e. compounds wherein the repeating units are linked to each other via ether bonds. Preferred perfluoropolyethers suitable for use in the present invention are those with repeating units of the type -[perfluoroalkandiyl-O]—, in particular —[CF₂—O]—, —[CF₂—CF₂—O]—, —[CF₂—CF₂—CF₂—O]—, and/or —[CF(CF₃)CF₂—O]—. Typical end groups of perfluoropolyether are for example CF₃O—, C₂F₅O—, and/or C₃F₇O—. Polytetrafluoroethylene oxide or polyhexafluoropropylene oxide can be mentioned as examples of perfluoropolyethers.

Compounds of formula (II) can for example be used to color common commercially available perflouropolyethers, such as e.g. perfluoropolyethers with average molecular weights (number average) in the range of 200 to 2000 g/mol and/or with a kinematic viscosity of 0.3 to 20.0 mm²/s, in particular 0.3 to 15 mm²/s at 25° C.

For the use according to the present invention, the compound of formula (II) and the compound to be colored are mixed. In the process, the compound of formula (II) dissolves in the liquid compounds defined above to a sufficient extent. Optionally, the dissolution can be accelerated by stirring or heating the mixture. If the compound of formula (II) is used to color a solid, it can be helpful to add the compound of formula (II) already during the production of the solid, e.g. prior to curing a polysiloxane. However, due to the sublimation tendency of compounds of formula (II), in particular azulene, it is also possible to place a solid material with a corresponding compound of formula (II) in a suitable closed container in which the compound of formula (II) penetrates the solid by way of diffusion.

Advantageously, according to the present invention it is not necessary to add a solubilizer which increases the solubility of the compound of formula (II) to the compound to be colored in addition to the compound of formula (II). Partially fluorinated hydrocarbons, i.e. hydrocarbons wherein only some hydrogen atoms have been replaced with fluorine, are known as such solubilizers, e.g. for introducing colorants into perfluorocarbon compounds. Surfactants with hydrophilic and hydrophobic portions are also suitable as solubilizers. Such compounds are usually not necessary and their use can be foregone.

The compound of formula (II), in particular azulene, is used in the compound to be colored in an amount sufficient to lead to the desired coloration. Typically, the amount of compound(s) of formula (II) is in the range of 0.001 mol/L to 1 mol/L, preferably in the range of 0.002 to 0.7 mol/L, and more preferred in the range of 0.002 to 0.5 mol/L, based on the amount of compound(s) to be colored selected from a siloxane, a perfluorocarbon compound, a perfluoroether and a perfluoroamine.

An intense coloration can be achieved by the use of a compound of formula (II) according to the present invention. Usually, the colored compounds show an absorption maximum in the visible range at about 550 to 600 nm, in particular 560 to 585 nm, and thus appear blue.

The solubility of compounds of formula (II) and in particular of azulene in siloxanes and perfluorinated compounds is of particular interest for applications in technology, research and medicine since those substances are often colorless and thus hard to identify visually. By coloring them with azulene, such phases, especially liquid phases which are hard to color such as silicone oil or perfluorinated liquids, including perfluorinated polyethers, are easier to detect. The detection can be accomplished visually or mechanically simply via the light absorption.

In the context of the present invention, a process for coloring a compound selected from a siloxane, a perfluorocarbon compound, a perfluoroether and a perfluoroamine as defined above is thus also provided, wherein the process comprises mixing a compound of formula (II), in particular azulene, with the compound to be colored. Apart from that, the above-mentioned definitions and preferred definitions, for example for the compound to be colored, the compound of formula (II) and the amounts to be used, apply to this process as well.

According to another aspect of the present invention, a composition is provided comprising a compound selected from a siloxane, a perfluorocarbon compound, a perfluoroether, a perfluoroamine and combinations thereof as a main component, based on the total weight of the composition, and azulene or an azulene derivative of formula (II), wherein the azulene or azulene derivative of formula (II) is dissolved in the compound selected from a siloxane, a perfluorocarbon compound, a perfluoroether, a perfluoroamine and mixtures thereof.

The above-mentioned definitions and preferred definitions, as given for the aspect of the use of the compound of formula (II) for coloring, apply to the compound of formula (II) as well as to the compound selected from a siloxane, a perfluorocarbon compound, a perfluoroether and a perfluoroamine.

As within the framework of the first aspect of the present invention regarding the use of a compound of formula (II) for coloring, the composition according to the present invention can also comprise a single compound of formula (II), in particular azulene. However, if needed, two or more different compounds of formula (II) can be used in admixture.

The composition can also comprise a single compound selected from a siloxane, a perfluorocarbon compound, a perfluoroether and a perfluoro amine, or a combination of two or more of these compounds. In the case of a combination, the sum of the weight percentages of the respective compounds has to represent the main component, based on the total weight of the composition.

As is evident to the person skilled in the art, the term “main component”, based on the total weight of the composition, means that the compound selected from a siloxane, a perfluorocarbon compound, a perfluoroether and a perfluoroamine, or a combination of two or more of these compounds, provides the highest weight proportion of the weight of all the components of the composition. Typically, the composition according to the present invention comprises the compound(s) selected from a siloxane, a perfluorocarbon compound, a perfluoroether and a perfluoroamine in an amount of 50 wt.-% or more, based on the total weight of the composition. Amounts of 75 wt.-% or more are preferred, 80 wt.-% or more are more preferred and 90 wt.-% or more are especially preferred. The composition can also consist of only the compound(s) of formula (II), in particular azulene, and the compound(s) selected from a siloxane, a perfluorocarbon compound, a perfluoroether and a perfluoroamine.

The compound(s) selected from a siloxane, a perfluorocarbon compound, a perfluoroether and a perfluoroamine which are used in the composition according to the present invention are preferably liquid at a temperature of 20° C. and a pressure of 1 atm (101325 Pa). The same equally applies to the composition as a whole.

The compound of formula (II), in particular azulene, is used in the composition in an amount sufficient to lead to the desired coloration. Typically, the amount of compound(s) of formula (II) is in the range of 0.001 mol/L to 1 mol/L, preferably in the range of 0.002 to 0.7 mol/L, and more preferred in the range of 0.002 to 0.5 mol/L, based on the amount of compound(s) to be colored selected from a siloxane, a perfluorocarbon compound, a perfluoroether and a perfluoroamine.

Advantageously, according to the present invention it is not necessary to add a solubilizer which increases the solubility of the compound of formula (II) to the compound to be colored in addition to the compound of formula (II). Partially fluorinated hydrocarbons, i.e. hydrocarbons wherein only some hydrogen atoms have been replaced with fluorine, are known as such solubilizers, e.g. for introducing colorants into perfluorocarbon compounds.

Surfactants with hydrophilic and hydrophobic portions are also suitable as solubilizers. Such compounds are usually not necessary and their use can be foregone.

An intense coloration can be achieved by the use of a compound of formula (II). Usually, the compositions of the present invention show an absorption maximum in the visible range at about 550 to 600 nm, in particular 560 to 585 nm, and thus appear blue. The extinction coefficient of the compound of formula (II), measured in chloroform, at the absorption maximum in the visible range lies for example in the range of 100 to 1000, in particular 200 to 500 L×mol⁻¹×cm⁻¹. Azulene as compound of formula (II) additionally exhibits the extraordinary property of fluorescing in its second excited state, while according to Kasha's rule, fluorescence otherwise occurs from the first excited state.

Such a coloration can for example be desired for compositions, which, without the presence of a compound of formula (II), show no or little capability to absorb light in the wave length range of about 380 to 780 nm and which appear transparent, in particular transparent liquids such as e.g. silicone oils or liquid perfluorinated compounds. According to the present invention, they can be provided as colored compositions which are therefore easier to detect. Due to the surprisingly high degree of solubility of compounds of formula (II), the transparency of the compounds is maintained in the process.

As will be explained below, one field of application for the compositions according to the present invention, for example the above-mentioned transparent, colored liquids, is medicine. For such applications, it is possible to additionally include one or more active ingredients in the composition such as an antibiotic. Optionally, the composition can also comprise other pharmaceutically acceptable components such as an antioxidant or an agent for adjusting viscosity.

Liquid siloxanes and perfluorinated compounds, such as the perfluorocarbon compounds, perfluoroethers and perfluoroamines mentioned above, and in particular silicone oils and liquid perfluorocarbon compounds, are for example used in ophthalmology. The compositions according to the present invention are excellently suited for such purposes since in addition to the advantages of such compounds, such as ease of handling and non-toxic properties, they are also colored and therefore easily visible to the surgeon. This facilitates, for instance, the residue-free removal of such compositions after they have served their purpose during surgery.

In this respect, another aspect of the present invention is directed to the composition according to the present invention for the use in the therapeutic or surgical treatment of the body of a human or an animal, in particular the eye, or for the use in a diagnostic method practiced on the body of a human or an animal, in particular the eye.

According to a preferred embodiment, the composition according to the present invention can be used in the surgical treatment of the eye of a human or an animal, such as retinal surgery and vitrectomy, e.g. in surgical procedures dealing with retinal detachment. During such a treatment, the composition according to the present invention can for example be administered as an infusion solution.

Within the framework of the surgical treatment of the eye, the compositions according to the present invention can for example be used as tamponade, in particular as vitreous tamponade or retinal tamponade, or as a tool in the intraoperative unfolding of the retina. Processes for the surgical treatment of the eye using siloxanes or perfluorinated compounds are known and described in the literature, reference can e.g. be made to WO 03/079927, DE4220882 or U.S. Pat. No. 4,490,351 and the references cited therein.

Compositions according to the present invention comprising one or more perfluoroalkanes, perfluorocycloalkanes or perfluoro aromatics as compound(s) selected from a siloxane, a perfluorocarbon compound, a perfluoroether and a perfluoroamine are especially suitable for use in the surgical treatment of the eye, in particular those wherein the phase to be colored consists of one of these compounds. Perfluorooctane (e.g. perfluoro-n-octane), perfluorodecalin, perfluorophenanthrene, and perfluoroethylcyclohexane are established in the field of ophthalmology and are therefore especially preferred.

Of course, the compositions according to the present invention comprising a compound of formula (II) and a compound selected from a siloxane, a perfluorocarbon compound, a perfluoroether and a perfluoroamine can also be used in other applications of siloxanes or perfluorinated compounds where the coloring with compound (II) can be advantageous. For instance, compositions according to the present invention comprising a silicone oil can be used as lubricants.

### EXAMPLES

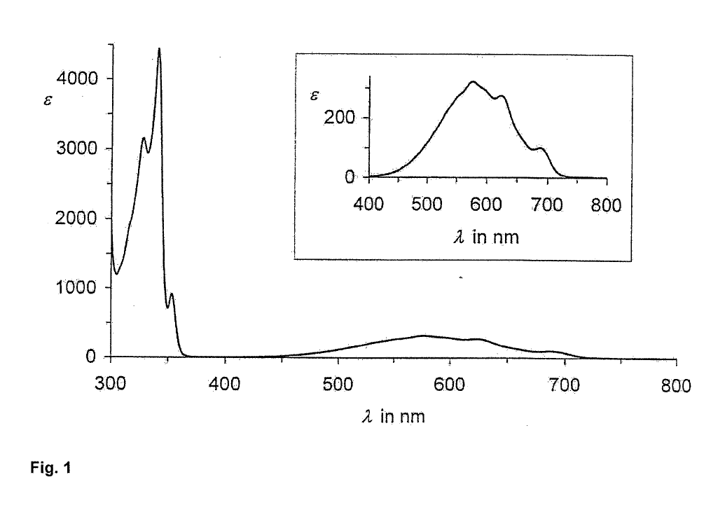

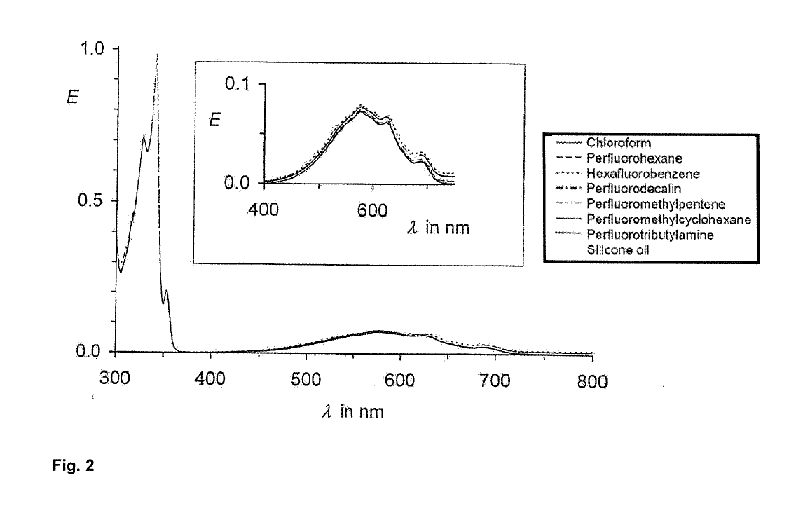

The saturation concentration of azulene (I) in different solvents was determined. The saturation concentration indicates at which concentration of the substance to be dissolved a saturated solution is formed in the respective solvent. It is thus an indicator of the maximum solubility of the substance in the solvent. In addition, UV/Vis spectra of the solutions were recorded. The devices Varian Cary 5000 and Bruins Omega 20 were used for measuring. The results are shown in FIGS. 1 and 2.

| | Saturation |
|---|---|
| Solvent | concentration |
| Silicone oil (Baysilon Grüssing 250° C.; Wacker | 0.11 mol · L⁻¹ |
| silicone oil AK | |
| 100, n = 70, Mₙ 5000) | |
| Perfluorohexane (RN 355-42-0) | 0.0027 mol · L⁻¹ |
| Perfluoromethylcyclohexane (RN 355-02-2) | 0.0034 mol · L⁻¹ |
| Perfluorodecalin (RN 306-94-5) | 0.0036 mol · L⁻¹ |
| Perfluoro-2-methyl-2-pentene (RN 1584-03-8) | 0.0057 mol · L⁻¹ |
| Perfluorobutylamine (RN 311-89-7) | 0.0026 mol · L⁻¹ |
| Hexafluorobenzene (RN 392-56-3) | 0.65 mol · L⁻¹ |
| Poly-1,1,2,3,3,3-hexafluoropropylene oxide | 0.0031 mol · L⁻¹ |
| (RN 69991-67-9; HT110, boiling point 110° C., | |
| M,, 580) | |

FIG. 1 shows the UV/Vis absorption spectrum of azulene (I) in chloroform. The extinction coefficient is 4446 L·mol⁻¹-cm⁻¹ at 340.6 nm or 323 L·mol⁻¹·cm⁻¹ at 578.4 nm.

FIG. 2 shows the standardized UV/Vis absorption spectra of azulene (I) in various media. The UV/Vis absorption spectrum of azulene (I) is only slightly affected by the listed media.

1. Use of azulene or an azulene derivative of the general formula (II): 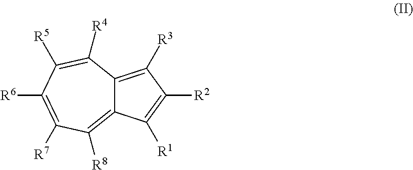
for coloring a compound selected from a siloxane, a perfluorocarbon compound, a perfluoroether and a perfluoro amine,
wherein in formula (II) the groups R¹ to R⁸ can be the same or different and are independently selected from a hydrogen atom, a halogen atom, a cyano group, a linear alkyl group, a group —(CH₂)ₙ-phenyl, a group —(CH₂)ₙ-pyridyl, a group —(CH₂)ₙ-thiophene, wherein n is an integer from 0 to 6, a naphthalene group, wherein one or two CH groups can be replaced with nitrogen atoms, or an anthracene group, wherein one or two CH groups can be replaced with nitrogen atoms,
wherein the linear alkyl group is an alkyl group with at least one and at most 37 C-atoms, wherein one to 10 CH₂ units can independently be replaced each with a carbonyl group, an oxygen atom, a sulfur atom, a selenium atom, a tellurium atom, a cis- or trans-CH═CH group, wherein one CH unit can also be replaced with a nitrogen atom, an acetylenic C≡C group, a divalent phenyl group (e.g. a 1,2-, 1,3- or 1,4-phenyl group), a divalent pyridine group, a divalent thiophene group, a divalent naphthalene group, wherein one or two CH groups can be replaced with nitrogen atoms, or a divalent anthracene group, wherein one or two CH groups can be replaced with nitrogen atoms,
wherein up to 12 individual hydrogen atoms of the CH₂ groups in an alkyl group can independently be replaced even on the same C-atom each with a halogen atom, a cyano group, or a linear alkyl chain with up to 18 C-atoms, wherein one to 6 CH₂ units can independently be replaced with a carbonyl group, an oxygen atom, a sulfur atom, a selenium atom, a tellurium atom, a cis- or trans-CH═CH group, wherein one CH unit can also be replaced with a nitrogen atom, an acetylenic C≡C group, a divalent phenyl group, a divalent pyridine group, a divalent thiophene group, a divalent naphthalene group, wherein one or two CH groups can be replaced with nitrogen atoms, or a divalent anthracene group, wherein one or two CH groups can be replaced with nitrogen atoms, and wherein up to 12 individual hydrogen atoms of the CH₂ groups in the alkyl chain which can serve as the substituent at the alkyl group can independently be replaced even on the same C-atom each with a halogen atom, a cyano group, or a linear alkyl chain with up to 18 C-atoms, wherein one to 6 CH₂ units can independently be replaced each with a carbonyl group, an oxygen atom, a sulfur atom, a selenium atom, a tellurium atom, a cis- or trans-CH═CH group, wherein one CH unit can also be replaced with a nitrogen atom, an acetylenic C≡C group, a divalent phenyl group, a divalent pyridine group, a divalent thiophene group, a divalent naphthalene group, wherein one or two CH groups can be replaced with nitrogen atoms, or a divalent anthracene group, wherein one or two CH groups can be replaced with nitrogen atoms,
and wherein two linear alkyl groups as defined above and located at different positions R¹ to R⁸ can optionally be linked to each other forming a ring.

2. Use according to claim 1, wherein the groups R¹ to R⁸ in formula (II) are independently selected from a hydrogen atom, C₁-C₆ alkyl, or (C₁-C₆ alkyl)-O—.

3. Use according to claim 1, wherein the compound of formula (II) is azulene.

4. Use according to claim 1, wherein the compound to be colored is a silicone oil.

5. Use according to claim 1, wherein the compound to be colored is a perfluoroalkane, a perfluorocycloalkane or a perfluoroaromatic.

6. Use according to claim 1, wherein the compound to be colored is a perfluoroether.

7. Composition comprising a compound selected from siloxane, a perfluorocarbon compound, a perfluoroether, a perfluoroamine and mixtures thereof as a main component, based on the total weight of the composition, and azulene or an azulene derivative of the formula (II), 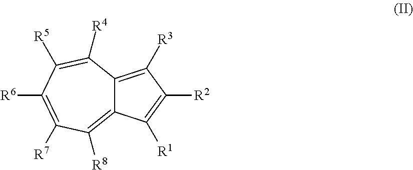
wherein in formula (II) the groups R¹ to R⁸ can be the same or different and are independently selected from a hydrogen atom, a halogen atom, a cyano group, a linear alkyl group, a group —(CH₂)ₙ-phenyl, a group —(CH₂)ₙ-pyridyl, a group —(CH₂)ₙ-thiophene, wherein n is an integer from 0 to 6, a naphthalene group, wherein one or two CH groups can be replaced with nitrogen atoms, or an anthracene group, wherein one or two CH groups can be replaced with nitrogen atoms,
wherein the linear alkyl group is an alkyl group with at least one and at most 37 C-atoms, wherein one to 10 CH₂ units can independently be replaced each with a carbonyl group, an oxygen atom, a sulfur atom, a selenium atom, a tellurium atom, a cis- or trans-CH═CH group, wherein one CH unit can also be replaced with a nitrogen atom, an acetylenic C≡C group, a divalent phenyl group, a divalent pyridine group, a divalent thiophene group, a divalent naphthalene group, wherein one or two CH groups can be replaced with nitrogen atoms, or a divalent anthracene group, wherein one or two CH groups can be replaced with nitrogen atoms,
wherein up to 12 individual hydrogen atoms of the CH₂ groups in an alkyl group can independently be replaced even on the same C-atom each with a halogen atom, a cyano group, or a linear alkyl chain with up to 18 C-atoms, wherein one to 6 CH₂ units can independently be replaced each with a carbonyl group, an oxygen atom, a sulfur atom, a selenium atom, a tellurium atom, a cis- or trans-CH═CH group, wherein one CH unit can also be replaced with a nitrogen atom, an acetylenic C≡C group, a divalent phenyl group, a divalent pyridine group, a divalent thiophene group, a divalent naphthalene group, wherein one or two CH groups can be replaced with nitrogen atoms, or a divalent anthracene group, wherein one or two CH groups can be replaced with nitrogen atoms, and wherein up to 12 individual hydrogen atoms of the CH₂ groups in the alkyl chain which can serve as the substituent at the alkyl group can independently be replaced also on the same carbon atom each with a halogen atom, a cyano group, or a linear alkyl chain with up to 18 C-atoms, wherein one to 6 CH₂ units can independently be replaced with a carbonyl group, an oxygen atom, a sulfur atom, a selenium atom, a tellurium atom, a cis- or trans-CH═CH group, wherein one CH unit can also be replaced with a nitrogen atom, an acetylenic C≡C group, a divalent phenyl group, a divalent pyridine group, a divalent thiophene group, a divalent naphthalene group, wherein one or two CH groups can be replaced with nitrogen atoms, or a divalent anthracene group, wherein one or two CH groups can be replaced with nitrogen atoms,
and wherein two linear alkyl groups as defined above and located at different positions R¹ to R⁸ can optionally be linked to each other forming a ring; and
wherein the azulene or azulene derivative of formula (II) is dissolved in the compound selected from a siloxane, a perfluorocarbon compound, a perfluoroether, a perfluoroamine and combinations thereof.

8. Composition according to claim 7, wherein the composition comprises the compound selected from a siloxane, a perfluorocarbon compound, a perfluoroether, a perfluoroamine and combinations thereof in an amount of 50 wt.-% or more, based on the total weight of the composition.

9. Composition according to claim 7, wherein the groups R¹ to R⁸ in formula (II) are independently selected from a hydrogen atom, C₁-C₆ alkyl, or (C₁-C₆ alkyl)-O—.

10. Composition according to claim 7, wherein the compound of formula (II) is azulene.

11. Composition according to claim 7, wherein the compound in which the azulene or azulene derivative is dissolved is a silicone oil.

12. Composition according to claim 7, wherein the compound in which the azulene or azulene derivative is dissolved is a perfluoroalkane, a perfluorocycloalkane or a perfluoroaromatic.

13. Composition according to claim 7, wherein the compound in which the azulene or azulene derivative is dissolved is a perfluoroether.

14. Use of a composition according to claim 7 in the surgical or therapeutic treatment of the eye of a human or an animal.

15. Use according to claim 14 in vitrectomy.

16-17. (canceled)
